(19) United States
(12) Patent Application Publication
(10) Pub. No.: US 2010/0204333 A1
(43) Pub. Date: Aug. 12, 2010

(54) Novel Pharmaceutical Modified Release Dosage Form Cyclooxygenase Enzyme Inhibitor

(75) Inventors: Rajesh Jain, New Delhi (IN); Kour Chand Jindal, New Delhi (IN); Sukhjeet Singh, New Delhi (IN); Munish Talwar, New Delhi (IN)

Correspondence Address: LADAS & PARRY LLP, 26 WEST 61ST STREET, NEW YORK, NY, 10023, US

(73) Assignee: PANACEA BIOTEC LTD., New Delhi (IN)

(21) Appl. No.: 11/988,860

(22) PCT Filed: Jul. 19, 2006
(86) PCT No.: PCT/IN2006/000258

§ 371 Date: Jan. 15, 2008

(30) Foreign Application Priority Data
Jul. 20, 2005 (IN) 1899/DEL/2005

(51) Int. Cl. A61K 31/18; A61P 25/00; A61P 25/06

(52) U.S. Cl. 514/605

(57) ABSTRACT

Pharmaceutical modified release dosage form comprising at least one cyclooxygenase enzyme inhibitor or its pharmaceutically acceptable salts, esters, prodrugs, solvates, hydrates, or derivatives thereof as active agent, with a pharmaceutically acceptable carrier for controlling the release of the cyclooxygenase enzyme inhibitor is provided. The dosage form preferably provides a release of not more than about 60% of the cyclooxygenase enzyme inhibitor in 1 hour and not less than about 75% of the cyclooxygenase enzyme inhibitor after 12 hours when tested in accordance with the dissolution method (I) described herein employing Distilled water with 2.0% Sodium lauryl sulphate as the dissolution medium or in accordance with the dissolution method (II) described herein employing pH 7.0 Phosphate buffer with 2.0% Sodium lauryl sulphate as the dissolution medium or in accordance with the dissolution method (III) described herein employing 0.001 N Hydrochloric acid with 1.0% Sodium lauryl sulphate as dissolution medium. Further, the pharmaceutical composition of the present invention when tested in a group of healthy humans preferably achieves a mean peak plasma concentration (Cₘₐₓ) after at least about 1 hour of administration of the dosage form. The present invention also provides process of preparing such dosage form compositions and prophylactic and/or therapeutic methods of using such dosage form.

### FIELD OF THE INVENTION

The present invention relates to pharmaceutical modified release dosage form comprising at least one cyclooxygenase enzyme inhibitor or its pharmaceutically acceptable salts, esters, prodrugs, solvates, hydrates, or derivatives thereof as active agent, with a pharmaceutically acceptable carrier for controlling the release of the cyclooxygenase enzyme inhibitor. Further, the pharmaceutical composition of the present invention provides for the administration of a therapeutically and/or prophylactically effective amount of the active agent. Furthermore, the dosage form preferably provides a release of not more than about 60% of the cyclooxygenase enzyme inhibitor in 1 hour and not less than about 75% of the cyclooxygenase enzyme inhibitor after 12 hours when tested in accordance with the dissolution method (I) described herein employing Distilled water with 2.0% Sodium lauryl sulphate as the dissolution medium or in accordance with the dissolution method (II) described herein employing pH 7.0 Phosphate buffer with 2.0% Sodium lauryl sulphate as the dissolution medium or in accordance with the dissolution method (III) described herein employing 0.001 N Hydrochloric acid with 1.0% Sodium lauryl sulphate as dissolution medium. Still further, the pharmaceutical composition of the present invention when tested in a group of healthy humans preferably achieves a mean peak plasma concentration (Cₘₐₓ) after at least about 1 hour of administration of the dosage form. The present invention also provides process of preparing such dosage form compositions and prophylactic and/or therapeutic methods of using such dosage form.

### BACKGROUND OF THE INVENTION

Cyclooxygenase-1 (COX-1) is an enzyme which is normally present in a variety of areas of the body, including sites of inflammation and the stomach. The COX-1 enzyme of the stomach produces certain chemical messengers (called prostaglandins) that ensure the natural mucus lining which protects the inner stomach. Common anti-inflammatory drugs like aspirin block the function of the COX-1 enzyme along with another enzyme, COX-2 (see below). When COX-1 enzyme is blocked, inflammation is reduced, but the protective mucus lining of the stomach is also reduced, that can cause stomach upset, ulceration, and bleeding from stomach and intestines. Cyclooxygenase-2 (COX-2) inhibitors are newly developed drugs for inflammation that selectively block the COX-2 enzyme. Blocking this enzyme impedes the production of the chemical messengers (prostaglandins) that cause the pain and swelling of arthritis inflammation. COX-2 inhibitors are a new class of nonsteroidal anti-inflammatory drugs (NSAIDs). Because they selectively block the COX-2 enzyme and not the COX-1 enzyme, these drugs are uniquely different from traditional NSAIDs. This selective action provides the benefits of reducing inflammation without irritating the stomach. These drugs pose an advantage in comparison to previous anti-inflammatory drugs because their mechanism of action carries nowhere near the risk of stomach ulceration and bleeding. The COX-2 inhibitors include celecoxib, rofecoxib, etoricoxib, valdecoxib, itacoxib, deracoxib and the like. Nonsteroidal anti-inflammatory drugs (NSAIDs) are commonly prescribed medications for the inflammation of arthritis and other body tissues, such as in tendinitis and bursitis. Examples of NSAIDs include aspirin, indomethacin, nimesulide, ketorolac, diclofenac, ibuprofen, naproxen, piroxicam, nabumetone, and the like. Nimesulide is a potent NSAID, presently used in the treatment of painful inflammatory conditions, due to rheumatoid arthritis, which also possesses antipyretic activity. Compared to other NSAIDs, nimesulide has a better therapeutic ratio, low gastrotoxicity and generally good tolerability. Nimesulide is a strongly hydrophobic substance that is practically insoluble in water (solubility in water at room temperature being 0.01 mg/ml).

Drug levels can be maintained above the lower level of the therapeutic plasma concentration for longer periods of time by administering larger doses of conventionally formulated dosage forms, but this approach might produce toxic effects due to high plasma concentration of the drug. Alternatively, another approach is to administer a drug at certain intervals of time, resulting in oscillating drug levels, the so-called peak and valley effect. This approach is generally associated with several potential problems, such as a large peak (toxic effect) and valley (non-active drug level) effect, and a lack of patient compliance leading to drug therapy inefficiency or failure. To overcome such issues, modified release compositions can be formulated with the objective of either releasing the drug in a sustained or controlled manner for an extended period of time or releasing a portion of the drug immediately followed by a sustained or controlled release of the drug. PCT publication bearing no. WO 95/14460 describes such compositions which initially release a burst of a therapeutic agent and then release the agent at an essentially constant rate for extended time period. Patients suffering from pain and/or inflammatory conditions primarily require high daily dosages of NSAIDs. In order to administer such high doses of NSAID only once a day, the release from the dosage form must be safe, predictable and reliable. Also the dosage form should be designed such that there is no sudden undesirable rise in plasma concentrations due to dose dumping. Moreover, the rate and extent of release and also the release pattern of the drug from the composition during in-vitro evaluation should correlate substantially to in-vivo performance of the composition.

U.S. Pat. No. 6,713,089 describes a quick release pharmaceutical composition for oral administration comprising a therapeutically and/or prophylactically active substance such as nimesulide and at least one pharmaceutically acceptable excipient, said active substance being defined by one of features such as when tested in accordance with the dissolution method USP XXIII Apparatus 2 employing 0.07 N hydrochloric acid as dissolution medium, at least 50% w/w of the active substance is dissolved within the first 20 minutes of the test. U.S. Pat. No. 6,638,535 pertains to a pharmaceutical pellet comprising a substantially homogenous mixture of a rapidly-acting hypnotic agent or a pharmaceutically acceptable salt thereof and a pellet forming carrier of microcrystalline cellulose, wherein the amount of said hypnotic agent and said pellet forming carrier is at least 90% of the pellet weight, said pellet having a particle size within the range of 0.85 to 2.0 mm and wherein said pellet exhibits a dissolution profile under U.S. Pharmacopoeia XXIII, Apparatus I, in a basket apparatus at 37° C., in 0.01N HCl medium and at 100 r.p.m., such that at 5 minutes from the start of the test, less than 60% of the hypnotic agent has been released from the pellet.

Another U.S. Pat. No. 6,599,529 discloses an oral pharmaceutical modified release multiple-units composition in unit dosage form for administration of a therapeutically and/or prophylactically effective amount of a NSAID, said unit dosage form comprising at least two NSAID-containing fractions; a first NSAID-containing fraction of multiple-units for quick release of the NSAID, wherein said fraction comprises an antacid or an alkaline agent and wherein the quick in-vitro release is such that, when subjecting the first NSAID-containing fraction to dissolution in USP XXIII <711> Apparatus 2, dissolution medium 900.0 ml, at 50 rpm. employing 0.07 N HCl as dissolution medium, at least 50% w/w of the NSAID is released within the first 20 min of the test; and a second NSAID-containing fraction of multiple-units in the form of coated delayed release multiple units for extended release of the NSAID, said units coated with a coating substantially water-insoluble, but water-diffusible and substantially pH-independent, wherein said second NSAID-containing fraction of multiple-units releases from about 6% to 30% of said NSAID within 0.5 hours upon dissolution testing by USP XXIII <711> Apparatus 2, dissolution medium comprising 750 ml of 0.1 N HCl for 1 hour followed by 250 ml of dissolution medium comprising trisodium phosphate dodecahydrate and 0.1 N sodium hydroxide in distilled water at 50 r.p.m., and wherein the release of said second NSAID-containing fraction is independent of the release of said first NSAID-containing fraction.

U.S. Pat. No. 6,086,920 describes disintegratable microspheres giving 100% aqueous dissolution in less than 30 minutes made from a composition comprising about 50% to about 90% of at least one bio-affecting agent such as nimesulide; about 2% to about 40% of at least one disintegrant; and about 5% to about 15% by weight of at least one spheronization aid. US publication no. 20050020613 pertains to a sustained-release oral dosage form comprising a subunit, wherein the subunit comprises an opioid analgesic and a sustained-release material, wherein the dissolution rate in-vitro of the subunit, when measured by standard USP Drug Release test of U.S. Pharmacopeia (2003)<724>, is less than about 10% within about 6 hours and at least about 60% within about 24 hours; less than about 10% within about 8 hours and at least about 60% within about 24 hours; less than about 10% within about 10 hours and at least about 60% within about 24 hours; or less than about 10% within about 12 hours and at least about 60% within about 24 hours; the dosage form providing a duration of therapeutic effect of about 24 hours. US publication no. 20030170303 describes an orally deliverable pharmaceutical composition comprising a therapeutically effective amount of a selective cyclooxygenase-2 inhibitory drug of low water solubility and one or more pharmaceutically acceptable polymers, wherein the composition provides an in vitro sustained-release dissolution profile following placement in a standard dissolution medium exhibiting release of about 5% to about 35% of the drug 2 hours after said placement; release of about 10% to about 85% of the drug 8 hours after said placement; and release of about 30% to about 90% of the drug 18 hours after said placement. However, still there exists a need to develop oral modified release pharmaceutical compositions comprising NSAID for prophylactic and/or therapeutic use, which can release the drug in a desired manner so as to maintain therapeutic levels of the drug in the plasma for extended period of time but without causing drug related toxicity, and which can be prepared in an easy and cost-effective manner.

The inventors of the present invention have done extensive research and conducted several experiments to alleviate the drawbacks existing in prior art to develop a dosage form by using different excipients and formulation methods for modifying the release rate of a cyclooxygenase enzyme inhibitor preferably a NSAID so as to obtain the desired in vitro and/or in vivo release characteristics for providing release of the active agent for an extended duration of time devoid of any substantial toxicity, thus demonstrating a significant advancement over the prior art.

### SUMMARY OF THE INVENTION

It is an objective of the present invention to provide modified release pharmaceutical dosage form which comprises at least one cyclooxygenase enzyme inhibitor preferably a NSAID or its pharmaceutically acceptable salts, esters, prodrugs, solvates, hydrates, or derivatives thereof as active agent, said cyclooxygenase enzyme inhibitor treated with at least one release controlling polymer, wherein the dosage form provides a release of not more than about 60% of the cyclooxygenase enzyme inhibitor in 1 hour and not less than about 75% of the cyclooxygenase enzyme inhibitor after 12 hours when tested in accordance with the dissolution method (I) described herein employing Distilled water with 2.0% Sodium lauryl sulphate as the dissolution medium or in accordance with the dissolution method (II) described herein employing pH 7.0 Phosphate buffer with 2.0% Sodium lauryl sulphate as the dissolution medium or in accordance with the dissolution method (III) described herein employing 0.001 N Hydrochloric acid with 1.0% Sodium lauryl sulphate as dissolution medium.

It is an objective of the present invention to provide modified release pharmaceutical dosage form which comprises at least one cyclooxygenase enzyme inhibitor preferably a NSAID or its pharmaceutically acceptable salts, esters, prodrugs, solvates, hydrates, or derivatives thereof as active agent preferably having a solubility in water of at least 0.001 mg/ml water at 25° C., said cyclooxygenase enzyme inhibitor treated with at least one release controlling polymer wherein the dosage form provides a release of not more than about 60% of the cyclooxygenase enzyme inhibitor in 1 hour and not less than about 75% of the cyclooxygenase enzyme inhibitor after 12 hours when tested in accordance with the dissolution method (I) described herein employing Distilled water with 2.0% Sodium lauryl sulphate as the dissolution medium or in accordance with the dissolution method (II) described herein employing pH 7.0 Phosphate buffer with 2.0% Sodium lauryl sulphate as the dissolution medium or in accordance with the dissolution method (III) described herein employing 0.001 N Hydrochloric acid with 1.0% Sodium lauryl sulphate as dissolution medium.

It is an objective of the present invention to provide modified release pharmaceutical dosage form which comprises nimesulide or its pharmaceutically acceptable salts, esters, prodrugs, solvates, hydrates, or derivatives thereof as active agent, treated with at least one release controlling polymer wherein the dosage form provides a release of not more than about 60% of the cyclooxygenase enzyme inhibitor in 1 hour and not less than about 75% of the cyclooxygenase enzyme inhibitor after 12 hours when tested in accordance with the dissolution method (I) described herein employing Distilled water with 2.0% Sodium lauryl sulphate as the dissolution medium or in accordance with the dissolution method (II) described herein employing pH 7.0 Phosphate buffer with 2.0% Sodium lauryl sulphate as the dissolution medium or in accordance with the dissolution method (III) described herein employing 0.001 N Hydrochloric acid with 1.0% Sodium lauryl sulphate as dissolution medium.

It is an objective of the present invention to provide modified release pharmaceutical dosage form which comprises at least one cyclooxygenase enzyme inhibitor preferably a NSAID more preferably nimesulide or its pharmaceutically acceptable salts, esters, prodrugs, solvates, hydrates, or derivatives thereof as active agent, said cyclooxygenase enzyme inhibitor treated with at least one release controlling polymer wherein the dosage form provides a release of not more than about 60% of the cyclooxygenase enzyme inhibitor in 1 hour and not less than about 75% of the cyclooxygenase enzyme inhibitor after 12 hours when tested in accordance with the dissolution method (I) described herein employing Distilled water with 2.0% Sodium lauryl sulphate as the dissolution medium or in accordance with the dissolution method (II) described herein employing pH 7.0 Phosphate buffer with 2.0% Sodium lauryl sulphate as the dissolution medium or in accordance with the dissolution method (III) described herein employing 0.001 N Hydrochloric acid with 1.0% Sodium lauryl sulphate as dissolution medium; and wherein the said dosage form when tested in a group of healthy humans achieves a mean peak plasma concentration (Cₘₐₓ) after at least about 1 hour of administration of the dosage form, preferably within 2-13 hours, most preferably within 2-8 hours of administration.

It is another objective of the present invention to provide process of preparation of the dosage form which comprises treating the cyclooxygenase enzyme inhibitor preferably a NSAID more preferably nimesulide or its pharmaceutically acceptable salts, esters, prodrugs, solvates, hydrates, or derivatives thereof with at least one release controlling polymer and optionally with one or more pharmaceutically acceptable carrier, and formulating it into the desired dosage form.

It is yet another objective of the present invention to provide method of using the dosage form for the treatment of cyclooxygenase enzyme mediated disorders and/or cyclooxygenase inhibitor indicated disorders which comprises administrating to a subject in need thereof a pharmaceutically effective amount of the cyclooxygenase enzyme inhibitor preferably a NSAID more preferably nimesulide as the active ingredient or its pharmaceutically acceptable salts, esters, prodrugs, solvates, hydrates, or derivatives thereof.

It is yet another objective of the present invention to provide use of the pharmaceutical composition for the preparation of a medicament for the treatment of cyclooxygenase enzyme mediated disorders and/or cyclooxygenase inhibitor indicated disorders which comprises administrating to a subject in need thereof a pharmaceutically effective amount of the cyclooxygenase enzyme inhibitor preferably a NSAID more preferably nimesulide as the active ingredient or its pharmaceutically acceptable salts, esters, prodrugs, solvates, hydrates, or derivatives thereof.

The modified release pharmaceutical compositions of the present invention intended for once-a-day, twice-a-day or thrice-a-day administration, preferably for once-a-day administration, releases the drug in a desired manner so as to maintain prophylactic and/or therapeutic levels of the active agent in the plasma for extended period of time devoid of any substantial drug related toxicity, and also can be prepared in an easy and cost-effective manner.

### DETAILED DESCRIPTION OF THE INVENTION

The present invention provides modified release pharmaceutical dosage form which comprises at least one cyclooxygenase enzyme inhibitor preferably a NSAID or its pharmaceutically acceptable salts, esters, prodrugs, solvates, hydrates, or derivatives thereof as active agent, said cyclooxygenase enzyme inhibitor treated with at least one release controlling polymer wherein the dosage form provides a release of not more than about 60% of the cyclooxygenase enzyme inhibitor in 1 hour and not less than about 75% of the cyclooxygenase enzyme inhibitor after 12 hours when tested in accordance with the dissolution method (I) described herein employing. Distilled water with 2.0% Sodium lauryl sulphate as the dissolution medium or in accordance with the dissolution method (II) described herein employing pH 7.0 Phosphate buffer with 2.0% Sodium lauryl sulphate as the dissolution medium or in accordance with the dissolution method (III) described herein employing 0.001 N Hydrochloric acid with 1.0% Sodium lauryl sulphate as dissolution medium.

In an embodiment, the present invention provides modified release pharmaceutical dosage form which comprises at least one cyclooxygenase enzyme inhibitor preferably a NSAID or its pharmaceutically acceptable salts, esters, prodrugs, solvates, hydrates, or derivatives thereof as active agent preferably having a solubility in water of at least 0.001 mg/ml water at 25° C., said cyclooxygenase enzyme inhibitor treated with at least one release controlling polymer wherein the dosage form provides a release of not more than about 60% of the cyclooxygenase enzyme inhibitor in 1 hour and not less than about 75% of the cyclooxygenase enzyme inhibitor after 12 hours when tested in accordance with the dissolution method (I) described herein employing Distilled water with 2.0% Sodium lauryl sulphate as the dissolution medium or in accordance with the dissolution method (II) described herein employing pH 7.0 Phosphate buffer with 2.0% Sodium lauryl sulphate as the dissolution medium or in accordance with the dissolution method (III) described herein employing 0.001 N Hydrochloric acid with 1.0% Sodium lauryl sulphate as dissolution medium. Preferably the NSAID used as the active agent is nimesulide or its pharmaceutically acceptable salts, esters, prodrugs, solvates, hydrates, or derivatives thereof.

In an embodiment, the composition of the present invention provides a release of not more than about 60% of the cyclooxygenase enzyme inhibitor in about 1 hour and not less than about 75% of the cyclooxygenase enzyme inhibitor after about 12 hours when tested by USP Apparatus Type II (Paddles) at 100 rpm, using 1000 ml of Distilled water with 2.0% Sodium lauryl sulphate as the dissolution medium maintained at about 37±0.5° C. In another embodiment, the composition of the present invention provides a release of not more than about 60% of the cyclooxygenase enzyme inhibitor in about 1 hour and not less than about 75% of the cyclooxygenase enzyme inhibitor after about 12 hours when tested by the USP Apparatus Type II (Paddles) at 75 rpm, using 2000 ml of 0.001 N Hydrochloric acid with 1.0% Sodium lauryl sulphate as dissolution medium maintained at about 37±0.5° C. In yet another embodiment, the composition of the present invention provides a release of not more than about 60% of the cyclooxygenase enzyme inhibitor in about 1 hour and not less than about 75% of the cyclooxygenase enzyme inhibitor after about 12 hours when tested by the USP Apparatus Type II (Paddles) at 100 rpm, using 1000 ml of pH 7.0 Phosphate buffer with 2.0% Sodium lauryl sulphate as the dissolution medium maintained about at 37±0.5° C.

In a further embodiment of the present invention, the composition of the present invention provides a release of not more than about 60% of the cyclooxygenase enzyme inhibitor in about 1 hour when tested by USP Apparatus Type II (Paddles) at 100 rpm, using 1000 ml of dissolution medium maintained at about 37±0.5° C., wherein the dissolution medium is any one selected from pH 7.4 phosphate buffer (according to the USP) or USP Simulated Intestinal Fluid or USP Simulated Gastric fluid or pH 4.5 Acetate buffer (according to the USP). The term ‘USP’ used anywhere in the entire specification refers to the ‘United States Pharmacopoeia’.

In another embodiment of the present invention, the modified release composition when tested in a group of healthy humans achieves a mean peak plasma concentration (Cₘₐₓ) after at least about 1 hour of administration of the dosage form, preferably within about 2-13 hours, most preferably within about 2-8 hours of administration of the dosage form. The in vivo study conducted in healthy humans may be in the fasted state or fed state.

In another embodiment of the present invention, the pharmaceutical dosage form comprises a plurality of particles, wherein each particle comprises cyclooxygenase enzyme inhibitor or its pharmaceutically acceptable salts, esters, prodrugs, solvates, hydrates, or derivatives thereof, at least one release controlling polymer and optionally one or more pharmaceutically acceptable carrier(s).

In a preferred embodiment, the cyclooxygenase enzyme inhibitor of the present invention is a NSAID. In a most preferred embodiment, the NSAID is nimesulide or its pharmaceutically acceptable salts, esters, prodrugs, solvates, hydrates, or derivatives thereof. In a further embodiment, the dosage form of the present invention additionally comprises at least one other active ingredient(s). The other active agent useful in the present may be any agent known to the art that can be administered in combination with a cyclooxygenase enzyme inhibitor such as an active agent(s) selected from but not limited to a group comprising antipyretics such as acetaminophen, antiallergics such as cetirizine or loratadine or fexofenadine, aldosterone receptor antagonists, antibiotics, various enzymes, antimuscarinic agents, anti-viral agents, protein kinase inhibitors, α2-adrenergic agonist, ACE inhibitors, opoid analgesics, steroids, leukotriene B₄(LTB₄) receptor antagonists, leukotriene A₄ (LTA₄) hydrolase inhibitors, 5-HT agonists, HMG CoA inhibitors, H₂ antagonists, antineoplastic agents, antiplatelet agents, thrombin inhibitors, decongestants, diuretics, sedating or non-sedating anti-histamines, inducible nitric oxide synthase inhibitors, opioids, analgesics, Helicobacter pylori inhibitors, bronchodilators, spasmolytics such as scopolamine or glucagon, muscle relaxants, proton pump inhibitors, isoprostane inhibitors, PDE4-inhibitors, other NSAIDs, selective or preferential COX-2 inhibitors, COX-1 inhibitors, expectorants such as bromohexine and pseudoephedrine, analgesics such as codeine and chlorzoxazone and mefenamic acid and tramadol, antiemetics, urinary acidifiers such as racemethionine, chondroitin, glucosamine, methyl sulfonyl methane (MSM), aspirin, antidepressants, antipsychotics, antimigraine agents, and the like or mixtures thereof.

In another embodiment of the present invention, the dosage form provides a relatively rapid rise in plasma concentration of the active agent to a first initial early mean peak plasma concentration (Cₘₐₓ₁) in about 0.2 to about 6 hours after oral administration of the dosage form, followed by a second mean peak plasma concentration (Cₘₐₓ₂) which occurs in about 7 to about 20 hours after oral administration of the dosage form, said dosage form providing effective plasma concentration of active agent for prophylactic or therapeutic use against cyclooxygenase enzyme mediated disorders for at least about 8 hours preferably for at least about 12 hours more preferably for at least about 16 to about 24 hours after administration to a subject preferably mammals more preferably humans, in need thereof.

The composition of the present invention is prepared by using formulation techniques aimed at modified release of the cyclooxygenase enzyme inhibitor in a manner such that the bioavailability of dosage form thus obtained is at least comparable to a conventional immediate release dosage form preferably administered in the fed state. The release of the cyclooxygenase enzyme inhibitor from the dosage form of the present invention is controlled in a manner by using pharmaceutically acceptable carrier such that therapeutically effective plasma concentration of the drug can be obtained without any undesirable side effects for an extended period of time thus leading to improved patient compliance. In an embodiment, the dosage form composition preferably disintegrates into a plurality of particles upon in-vivo administration and gets substantially distributed throughout the gastrointestinal tract (GIT) independent of gastric emptying time and/or rate and/or motility thus preventing the high concentrations of drug in the GIT. In an embodiment, the dosage form comprises nimesulide as the active ingredient in at least 10% by weight of the dosage form. In another embodiment, the modified release dosage form of the present invention is in the extended release form, sustained release form, timed release form, pulsatile release form, prolonged release form, delayed release form or a combination of any such release forms. In a preferred embodiment, the modified release form is in the form of a combination of immediate release form and extended release form.

The cyclooxygenase enzyme inhibitor used in the composition of the present invention is selected from but not limited to the group comprising of lornoxicam, diclofenac, nimesulide, ibuprofen, piroxicam, naproxen, ketoprofen, tenoxicam, flosulide ibuprofen, indomethacin, aceclofenac, indometacin, nabumetone, acemetacin, momiflurnate, meloxicam, flurbiprofen, tiaprofenic acid, proglumetacin, mefenamic acid, fenbufen, etodolac, tolfenamic acid, sulindac, phenylbutazone, fenoprofen, tolmetin, acetylsalicylic acid, dexibuprofen, paracetamol, and pharmaceutically acceptable salts, complexes and/or prodrugs thereof and mixtures thereof. In an embodiment, the active agent used in the present invention is a COX-II inhibitor selected from but not limited to a group comprising celecoxib, rofecoxib, valdecoxib, etoricoxib, parecoxib, itacoxib, deracoxib and the like; their tautomeric forms, analogues, isomers, polymorphs, solvates, prodrugs or salts thereof. In an embodiment, the cyclooxygenase enzyme inhibitor used in the present invention as active agent also acts as a lipoxygenase inhibitor, such as, for example licofelone. The active agent(s) is one or more NSAIDs, one or more COX-II inhibitors or mixtures thereof. In an embodiment of the present invention, the active agent is in the micronized form.

The release controlling polymer of the present invention comprises a polymeric material selected from but not limited to the group comprising pH dependent polymers such as alginates or methacrylic acid polymers; pH independent polymers such as carbomers; soluble or insoluble polymers; swellable polymers; hydrophilic polymers; hydrophobic polymers; ionic polymers such as sodium alginate, carbomer, calcium carboxymethylcellulose or sodium carboxymethylcellulose; non-ionic polymers such as hydroxypropyl methylcellulose; synthetic or, natural polysaccharide selected from the group comprising alkylcelluloses, hydroxyalkyl celluloses, cellulose ethers, cellulose esters, nitrocelluloses, dextrin, agar, carrageenan, pectin, furcellaran, starch and starch derivatives, and mixtures thereof; cellulosic polymer, methacrylate polymer, polyvinylpyrrolidone (PVP), alginate, polyvinylpyrrolidone-polyvinylacetate polymer (PVP-PVA) copolymer, ethylcellulose, cellulose acetate, cellulose propionate (lower, medium or higher molecular weight), cellulose acetate propionate, cellulose acetate butyrate, cellulose acetate phthalate, cellulose triacetate, poly(alkyl methacrylate), poly(isodecyl methacrylate), poly(lauryl methacrylate), poly(phenyl methacrylate), poly(alkyl acrylate), poly(octadecyl acrylate), poly(ethylene), poly(alkylene), poly(alkylene oxide), poly(alkylene terephthalate), poly(vinyl isobutyl ether), poly(vinyl acetate), poly(vinyl chloride) and polyurethane or a mixture thereof used either alone or in combination thereof. In a further embodiment, the dosage form of the present invention additionally comprises a gum selected from but not limited to a group comprising xanthan gum, guar gum, gum arabic, carrageenan gum, karaya gum, locust bean gum, acacia gum, tragacanth gum, agar and the like or mixtures thereof. In another embodiment, the dosage form of the present invention additionally comprises at least one surfactant selected from a group comprising anionic surfactants, cationic surfactants, non-ionic surfactants, zwitterionic surfactants or mixtures thereof. In yet another embodiment, the dosage form of the present invention additionally comprises at least one complexing agent such as cyclodextrin selected from a group comprising but not limited to alpha-cyclodextrin, beta-cyclodextrin, betahydroxy-cyclodextrin, gamma-cyclodextrin, and hydroxypropyl beta-cyclodextrin, or the like.

The pharmaceutically acceptable carrier(s) used in the composition of the present invention are selected from but not limited to a group of excipients generally known to persons skilled in the art e.g. diluents such as lactose, mannitol, sorbitol, starch, microcrystalline cellulose, xylitol, fructose, sucrose, dextrose, dicalcium phosphate, calcium sulphate; disintegrants; binders; fillers; bulking agent; organic acid(s); colorants; stabilizers; preservatives; lubricants; glidants; chelating agents; vehicles; bulking agents; stabilizers; preservatives; hydrophilic polymers; solubility enhancing agents such as glycerin, various grades of polyethylene oxides, transcutol and glycofurol; tonicity adjusting agents; local anesthetics; pH adjusting agents; antioxidants; osmotic agents; chelating agents; viscosifying agents; wetting agents; emulsifying agents; acids; sugar alcohol; reducing sugars; non-reducing sugars and the like used either alone or in combination thereof. The disintegrants used in the present invention include but not limited to starch or its derivatives, partially pregelatinized maize starch (Starch 1500®), croscarmellose sodium, sodium starch glycollate, and the like used either alone or in combination thereof. The lubricants used in the present invention include but not limited to talc, magnesium stearate, calcium stearate, stearic acid, hydrogenated vegetable oil and the like used either alone or in combination thereof. The vehicles suitable for use in the present invention can be selected from but not limited to a group comprising dimethylacetamide, dimethylformamide and dimethylsulphoxide of N-methylpyrrolidone, benzyl benzoate, benzyl alcohol, ethyl oleate, polyoxyethylene glycolated castor oils (commercially available as Cremophor® EL), polyethylene glycol MW 200 to 6000, propylene glycol, hexylene glycols, butylene glycols and glycol derivatives such as polyethylene glycol 660 hydroxystearate (commercially available as Solutrol® HS15). In another embodiment of the present invention, the compositions may additionally comprise an antimicrobial preservative such as Benzyl alcohol preferably at a concentration of 2.0% v/v of the composition. In an embodiment of the present invention, the composition may additionally comprise a conventionally known antioxidant such as ascorbyl palmitate, butylhydroxyanisole, butylhydroxytoluene, propyl gallate and α-tocopherol.

It is another objective of the present invention to provide process of preparation of the dosage form which comprises treating the cyclooxygenase enzyme inhibitor preferably a NSAID more preferably nimesulide or its pharmaceutically acceptable salts, esters, prodrugs, solvates, hydrates, or derivatives thereof with at least one release controlling polymer and optionally with one or more pharmaceutically acceptable carrier, and formulating it into the desired dosage form.

The pharmaceutical dosage form composition of the present invention is preferably formulated as an oral dosage form such as tablets, capsules, patches and the like. In an embodiment, the composition of the present invention is in the form of tablets. The tablets can be prepared by either direct compression, dry compression (slugging), or by granulation. The granulation technique is either aqueous or non-aqueous. The non-aqueous solvent used is selected from a group comprising ethanol, isopropyl alcohol or methylene chloride. In an embodiment, the compositions of the present invention are in the form of compressed tablets, molded tablets, or products prepared by extrusion or film cast technique, and the like.

In another embodiment of the present invention is provided a method of using the dosage form for the treatment of cyclooxygenase enzyme mediated disorders and/or cyclooxygenase inhibitor indicated disorders which comprises administrating to a subject preferably mammals more preferably humans, in need thereof a pharmaceutically effective amount of the cyclooxygenase enzyme inhibitor preferably a NSAID more preferably nimesulide as the active ingredient or its pharmaceutically acceptable salts, esters, prodrugs, solvates, hydrates, or derivatives thereof. In yet another embodiment, the present invention provides use of the pharmaceutical composition for the preparation of a medicament for the treatment of cyclooxygenase enzyme mediated disorders and/or cyclooxygenase inhibitor indicated disorders which comprises administrating to a subject preferably mammals more preferably humans, in need thereof a pharmaceutically effective amount of the cyclooxygenase enzyme inhibitor preferably a NSAID more preferably nimesulide as the active ingredient or its pharmaceutically acceptable salts, esters, prodrugs, solvates, hydrates, or derivatives thereof.

In another embodiment of the present invention is provided an use of the dosage form for the treatment of cyclooxygenase enzyme mediated disorders and cyclooxygenase inhibitor indicated disorders which comprises administrating to a subject preferably mammals more preferably humans, in need thereof a pharmaceutically effective amount of nimesulide. In a further embodiment of the present invention, is provided an use of the dosage form for the management or treatment of particularly pain and/or inflammation associated with osteoarthritis; dental extraction or surgery; saphenectomy or inguinal hernioplasty; haemorrhoidectomy; acute musculoskeletal injury; ear, nose or throat disorders; gynecological disorders; cancer pain; alzheimer's disease; thrombophlebitis; urogenital disorders; bursitis or tendonitis; morning stiffness associated with rheumatoid arthritis, and the like. The analgesic and anti-inflammatory composition of the present invention is very useful in mammals particularly in humans for the treatment of acute painful conditions like post-operative trauma, pain associated with cancer, sports injuries, migraine headache, neurological pain and pain associated with sciatica and spondylitis, arthritis, and the like.

In a further embodiment of the present invention is provided an use of the dosage form composition comprising a cyclooxygenase enzyme inhibitor such as a NSAID particularly nimesulide for the management, prophylaxis or treatment of cyclooxygenase enzyme mediated disorders and cyclooxygenase inhibitor indicated disorders particularly pain and/or inflammation associated with osteoarthritis, ligamentous pain, bursitis, tendinitis, low back pain, postoperative pain, dental extraction or surgery; saphenectomy or inguinal hernioplasty; haemorrhoidectomy; acute musculoskeletal injury; ear, nose or throat disorders; gynecological disorders; cancer pain; alzheimer's disease; thrombophlebitis; urogenital disorders; bursitis or tendonitis; morning stiffness associated with rheumatoid arthritis, idiopathic pain, myofascial pain, osteoarthritis, neuropathic pain, fibromyalgia and inflammatory pain states such as rheumatoid arthritis and osteoarthritis. Neuropathic pain includes pain such as pain secondary to injury to nerves and includes postherpetic neuralgia, diabetic neuropathy, postamputation pain, mono- and polyneuropathies, radiculopathy, central pain, shingles, trigeminal neuralgia, temporomandibular joint disorder; cancer pain; chronic pain; acute pain; breakthrough pain sympathetically mediated pain, Raynaud's disease, CPS (Chronic Pain Syndrome); tension and migraine headache, stump pain, polyarteritis nodosa, osteomyelitis, bums involving nerve damage, AIDS related pain syndromes, and connective tissue disorders, such as systemic lupus erythematosus, systemic sclerosis, polymyositis, and dermatomyositis, other degenerative joint disorders and the like.

In an embodiment of the present invention, the dosage form compositions comprising nimesulide as active agent when tested in a group of at least twelve healthy humans achieves a mean peak plasma concentration (Cₘₐₓ) of nimesulide in the range of about 3-24 μg/ml, preferably in the range of about 6-12 μg/ml. In yet another embodiment, the dosage form comprises about 5 to about 400 mg of nimesulide and at least one release controlling polymer; said oral dosage form providing a mean Cₘₐₓ in the range of about 3-24 μg/ml achieved in a mean time (Tₘₐₓ) in the range of about 2-8 hours; said dosage form providing a therapeutic effect for at least about 8 to about 24 hours after oral administration, intended for once-a-day, twice-a-day or thrice-a-day administration. In a preferred embodiment, the dosage form according to the present invention is intended for once-a-day dosing.

In an embodiment, the dosage form of the present invention comprising nimesulide as the active agent provides an in-vitro dissolution of from about 5% to about 50% of nimesulide released after 1 hour; from about 40% to about 85% of nimesulide released after 6 hours; and not less than about 70% of nimesulide released after 12 hours when tested by the USP Apparatus Type II (Paddles) at 100 rpm using 1000 ml of Distilled water with 2.0% Sodium lauryl sulphate as dissolution medium maintained at about 37±0.5° C. temperature. In a further embodiment, the pharmaceutical dosage form of the present invention provides an in-vitro release of at least about 0.5% to about 15% of the active ingredient beyond 12 hours in the said dissolution medium under the said conditions.

In an embodiment of the present invention, the compositions of the present invention when tested in vivo exhibits a Cₘₐₓ (peak plasma concentration of the drug) of about 0.5-30 μg/ml and/or a Tₘₐₓ (time to reach peak plasma concentration) of about 1-12 hours.

The composition of the present invention can be formulated into a dosage form selected from the group consisting of oral solid dosage forms, liquid dispersions, oral suspensions, gels, aerosols, ointments, creams, fast melt formulations, rapidly disintegrating formulations, mucoadhesive formulations, gastroretentive formulations, lyophilized formulations, or the like.

### Dissolution Study Method

The dissolution study method (I) in accordance with the present invention has the following parameters:

| | |
|---|---|
| Dissolution medium | Distilled water with 2.0% Sodium |
| | Lauryl Sulphate |
| Dissolution medium volume | 1000 ml |
| Apparatus | Paddle (USP Type II) |
| Paddle Speed | 100 rpm |
| Temperature of dissolution medium | 37° C. ± 0.5° C. |

In an embodiment, the dissolution profile of the composition as described in Example-1 hereinafter comprising nimesulide as active agent is as follows:

| | | Dissolution profile |
|---|---|---|
| S. No. | Time (hours) | (% drug release) |
| 1 | 0.25 | 23.79 |
| 2 | 0.5 | 27.66 |
| 3 | 1 | 31.85 |
| 4 | 2 | 41.03 |
| 5 | 4 | 58.16 |
| 6 | 6 | 70.93 |
| 7 | 8 | 87.89 |
| 8 | 10 | 94.32 |
| 9 | 12 | 97.56 |

The dissolution study method (II) of the present invention has the following parameters:

| | |
|---|---|
| Dissolution medium | pH 7.0 Phosphate buffer with 2.0% |
| | Sodium Lauryl Sulphate |
| Dissolution medium volume | 1000 ml |
| Apparatus | Paddle (USP Type II) |
| Paddle Speed | 100 rpm |
| Temperature of dissolution medium | 37° C. ± 0.5° C. |

In an embodiment, the dissolution profile of the composition as described in Example-6 hereinafter comprising nimesulide as active agent is as follows:

| | | Dissolution profile |
|---|---|---|
| S. No. | Time (hours) | (% drug release) |
| 1 | 0.25 | 21.20 |
| 2 | 0.5 | 26.27 |
| 3 | 1 | 30.24 |
| 4 | 2 | 38.71 |
| 5 | 4 | 57.97 |
| 6 | 6 | 74.02 |
| 7 | 8 | 87.40 |
| 8 | 10 | 95.59 |
| 9 | 12 | 97.46 |

The dissolution study method (III) of the present invention has the following parameters:

| | |
|---|---|
| Dissolution medium | 0.001 N Hydrochloric acid with |
| | 1.0% Sodium Lauryl Sulphate |
| Dissolution medium volume | 1000 ml |
| Apparatus | Paddle (USP Type II) |
| Paddle Speed | 75 rpm |
| Temperature of dissolution medium | 37° C. ± 0.5° C. |

In an embodiment, the dissolution profile of the composition as described in Example-4 hereinafter comprising naproxen as active agent is as follows:

| | | Dissolution profile |
|---|---|---|
| S. No. | Time (hours) | (% drug release) |
| 1 | 0.25 | 0.00 |
| 2 | 0.5 | 0.00 |
| 3 | 1 | 15.11 |
| 4 | 2 | 28.71 |
| 5 | 4 | 37.85 |
| 6 | 6 | 44.32 |
| 7 | 8 | 56.11 |
| 8 | 10 | 65.50 |
| 9 | 12 | 77.67 |

Illustrated below are methods to carry out the in-vitro dissolution study of nimesulide. A similar dissolution method for other cyclooxygenase inhibitor can be used by making the necessary modifications specific to the properties of the active ingredient and the specific drug release (dissolution) medium used in the in-vitro study. Further, the dissolution methods may be modified depending on the composition and volume of dissolution medium used and, the type and speed of the Apparatus used to conduct the dissolution study.

Dissolution method (1): The drug release was measured and analyzed by HPLC with UV detector. The reagents used for performing the dissolution study comprise Sodium Lauryl Sulphate AR, Methanol AR and Distilled water. The Dissolution Medium was prepared as follows: 20 g of Sodium lauryl sulphate is dissolved in sufficient purified water and is made up to the 1000 ml with distilled water.

Dissolution Procedure: The dissolution apparatus is set by programming the temperature, rotation and run time at 37° C.±0.5° C., 100 rpm and 1 hour, 4 hour and 12 hours respectively. 1000 ml of 2.0% w/v of Sodium lauryl sulfate as Dissolution Medium is placed in each of the six vessels of the dissolution apparatus. The apparatus is assembled and the Dissolution Medium is equilibrated to 37° C.±0.5° C. and the thermometer is removed. One unit dosage is placed in each of the six vessels. Rotation of the paddle is started at the speed of 100 rpm for 12 hours. Aliquots (each of 10 ml) are withdrawn, and successively replaced with equal volumes of fresh Dissolution Medium, at the desired interval periods from each of the six vessels and the step is proceeded as given under ‘Test preparation’.

Standard preparation: About 80.0 mg of Nimesulide WS (working standard) is weighed and transferred accurately into a 100 ml volumetric flask. Nimesulide is dissolved and the volume is made up with Methanol. 5.0 ml of resulting solution is transferred to a 100 ml volumetric flask. The volume is made up with the Dissolution Medium followed by mixing.

Test preparation: Each of the dissolution samples withdrawn through 0.45 μm membrane filter (Millipore HVLP type) is filtered discarding first 5.0 ml of the filtrate. 2.0 ml of the filtrate is transferred to 10 ml volumetric flask. The volume is made up to the mark with Dissolution Medium followed by mixing.

Procedure: The test preparations (single injection) are separately injected into the chromatograph after filtering through 0.45 μm membrane filter. The chromatograms are recorded and the peak responses of Nimesulide peak are compared in terms of area in both standard and test preparations. The quantity of Nimesulide released in percent (%) with respect to claimed values in the present test preparations withdrawn at different intervals is calculated using the below mentioned formulae:

$$After 1 hour \text{:} = \frac{{A}_{T 1}}{{A}_{S}} \times \frac{{W}_{S}}{100} \times \frac{5}{100} \times \frac{1000}{C} \times \frac{10}{2} \times \frac{P}{100} \times 100$$

$$After 4 hour \text{:} = \frac{{A}_{T 4}}{{A}_{S}} \times \frac{{W}_{S}}{100} \times \frac{5}{100} \times \frac{1000}{C} \times \frac{10}{2} \times \frac{P}{100} \times 100 + {CR}_{4}$$

$$After 12 hours \text{:} = \frac{{Ab}_{12}}{{Ab}_{S}} \times \frac{{W}_{S}}{100} \times \frac{5}{100} \times \frac{1000}{C} \times \frac{10}{2} \times \frac{P}{100} \times 100 + {CR}_{12}$$

Where,

Ab₁=Area of peak due to Nimesulide in test preparation after 1 hour.
Ab₄=Area of peak due to Nimesulide in test preparation after 4 hours.
Ab₁₂=Area of peak due to Nimesulide in test preparation after 12 hours.
A_{S}=Average area of peak due to Nimesulide in standard preparation.
W_{S}=Weight of Nimesulide working standard taken (in mg).
P=Potency of Nimesulide working standard (in w/w).
C=Claim value of Nimesulide in each unit dosage (i.e. 200 mg).
CR₁₂=Corrected release of Nimesulide, in %, for 4^{th} and 12^{th}, calculated as given below:

$${CR}_{4} = \frac{\% Release at {1}^{st} hour}{1000} \times 10$$

$${CR}_{12} = \frac{{CR}_{4} + \% Release at {1}^{st} hour}{1000} \times 10.$$

Dissolution method (I): The drug release was measured and analyzed by HPLC with UV detector. The reagents used for performing the dissolution study comprise Sodium lauryl sulphate AR grade, Sodium hydroxide AR grade, Potassium phosphate AR grade and Distilled water.

Preparation of Dissolution medium (2% SLS in phosphate pH 7.0): Sodium hydroxide solution was prepared by dissolving 1.605 g of Sodium hydroxide in sufficient water to produce 1000 ml. Potassium phosphate solution was prepared by dissolving 5.444 g of Potassium dihydrogen orthophosphate phosphate in sufficient water to produce 1000 ml. 120 ml of Sodium hydroxide solution, 250 ml of Potassium phosphate solution and 20.0 g of Sodium lauryl sulphate were mixed in sufficient water to produce 1000 ml.

Dissolution procedure (replacement method): The dissolution apparatus was set by programming temperature, rotation and sampling intervals at 37° C., 100 rpm, and 1 hour, 4 and 12 hours, respectively. 1000 mL of the dissolution medium was placed in each of the six vessels of the dissolution apparatus. The apparatus was assembled and the dissolution medium equilibrated to 37° C.±0.5° C. One unit dose was placed in each of the six vessels and rotation of the paddle at the speed of 100 rpm was started and continued for 12 hours. The aliquots (each of 10 mL) were withdrawn at the interval period of 1 hour, 4 and 12 hours and successively replace with equal volumes of fresh dissolution medium at the interval period of 1 hour and 4 hours to each of the six vessels and proceed as given under test preparations.

Standard preparation: About 80.0 mg of Nimesulide working standard was weighed accurately and transferred into a 100 mL volumetric flask. The same was dissolved and volume made up with methanol. 5 mL of the resulting solution was transferred to a 100 mL volumetric flask and the volume made up with dissolution medium and mixed.

Test preparations: Each of the dissolution samples withdrawn were filtered through 0.45 μm membrane filter (Millipore HVLP type), discarding first 5 mL of the filtrate. 2 mL of the filtrate was transferred into a 10 mL volumetric flask and the volume made up with dissolution medium, and mixed.

Procedure: The test preparations (single injection) were separately injected into the chromatograph, after filtering through 0.45 μm membrane filter (Millipore HVLP type). The chromatograms were recorded and the peak responses of Nimesulide peak, in terms of area, in test preparation were compared. The quantity of Nimesulide released, in %, with respect to claim value, in each of the test preparations, withdrawn at different cumulative intervals, were calculated using the following formulae:

$$After 1 hour = \frac{{A}_{T 1}}{{A}_{S}} \times \frac{{W}_{S}}{100} \times \frac{5}{100} \times \frac{1000}{C} \times \frac{10}{2} \times \frac{P}{100} \times 100$$

$$After 4 hours = ( \frac{{A}_{T 4}}{{A}_{S}} \times \frac{{W}_{S}}{100} \times \frac{5}{100} \times \frac{1000}{C} \times \frac{10}{2} \times \frac{P}{100} \times 100 ) + {CR}_{4}$$

$$After 12 hours = ( \frac{{A}_{T 12}}{{A}_{S}} \times \frac{{W}_{S}}{100} \times \frac{5}{100} \times \frac{1000}{C} \times \frac{10}{2} \times \frac{P}{100} \times 100 ) + {CR}_{12}$$

Where,
A_{T1}=Area of peak due to Nimesulide in test preparation after 1 hour.
A_{T4}=Area of peak due to Nimesulide in test preparation after 4 hours.
A_{T12}=Area of peak due to Nimesulide in test preparation after 12 hours.
A_{S}=Average area of peak due to Nimesulide in standard preparation.
W_{S}=Weight of Nimesulide working standard taken (in mg)
P=Potency of Nimesulide working standard (in % w/w).
C=Claim value of Nimesulide in each tablet (i.e. 200 mg).
CR_{8,12}=Corrected release of Nimesulide, in %, for 4^{th} and 12^{th} hour, calculated as given below:

$${CR}_{4} = \frac{\% Release at {1}^{st} hour}{1000} \times 10$$

$${CR}_{12} = {CR}_{8} + ( \frac{\% Release at {4}^{th} hour}{1000} \times 10 )$$

Dissolution method (III): The drug release was measured and analyzed by UV-Spectroscopy using a UV/VIS Spectrophotometer Perkin Elmer Lambda 20 or equivalent. The reagents used for performing the dissolution study comprise Concentrated Hydrochloric Acid AR, Methanol AR, Sodium Lauryl Sulphate AR and Distilled water. The Dissolution Medium was prepared as follows: 0.17 ml of concentrated Hydrochloric acid is diluted in sufficient distilled water in a 2000 ml volumetric flask. 20 g of Sodium Lauryl Sulphate is then added and is made up to the volume with distilled water.

Dissolution Procedure: The dissolution apparatus is set by programming the temperature, rotation and run time at 37° C.±0.5° C., 75 rpm and 12 hours respectively. 2000 ml of dissolution Medium is placed in each of the six vessels of the dissolution apparatus. The apparatus is assembled and the dissolution Medium is equilibrated to 37° C.±0.5° C. One unit dosage is placed in each of the six vessels. Rotation of the paddle is started at the speed of 75 rpm for 12 hours. Aliquots (each of 10 ml) are withdrawn, and successively replaced with equal volumes of fresh Dissolution Medium, at the desired interval periods from each of the six vessels and the step is proceeded as given under ‘Test preparation’.

Standard preparation: About 100 mg of Nimesulide WS (working standard) is weighed and transferred accurately into a 100 ml volumetric flask. Nimesulide is dissolved and the volume is made up with Methanol. 2.0 ml of resulting solution is transferred to a 100 ml volumetric flask and 18 ml of Methanol is added. The volume is made up with the dissolution Medium followed by mixing.

Test preparation: Each of the dissolution samples withdrawn through 0.45 μm membrane filter is filtered discarding first few ml of the filtrate. 5.0 ml of the filtrate is transferred to 10 ml volumetric flask and 2.0 ml of Methanol is added for the samples withdrawn after 1 hour and 4 hours. 5.0 ml of the filtrate is transferred to 25 ml volumetric flask and 5.0 ml of Methanol is added for the samples withdrawn after 12 hours. The volume is made up to the mark with dissolution Medium.

Procedure: The absorbance of each of the Standard preparation and Test preparations withdrawn at different intervals using UV/VIS spectrophotometer at about 298 nm is measured by using Methanol and dissolution Medium (20:80) as a blank. The quantity of Nimesulide released in percent with respect to claimed values in the present Test preparations withdrawn at different intervals is calculated using the below mentioned formulae:

$$After 1 hour \text{:} = \frac{{Ab}_{T}}{{Ab}_{S}} \times \frac{{W}_{S}}{100} \times \frac{2}{100} \times \frac{2000}{C} \times \frac{10}{5} \times \frac{P}{100} \times 100$$

$$After 12 hours \text{:} = \frac{{Ab}_{T}}{{Ab}_{S}} \times \frac{{W}_{S}}{100} \times \frac{2}{100} \times \frac{2000}{C} \times \frac{25}{5} \times \frac{P}{100} \times \frac{2000}{1980} \times 100$$

Where,

Ab_{T}=Absorbance of test preparation.
Ab_{S}=Absorbance of standard preparation.
W_{S}=Weight of Nimesulide WS taken (in mg).
P=Potency of Nimesulide WS (in % w/w).
C=Claim value of Nimesulide in each unit dosage (i.e. 200 mg).

The influences of various process parameters on the Dissolution Rate of the cyclooxygenase enzyme inhibitor dosage form composition of the present invention were evaluated. Investigations by the inventors have indicated that the dissolution rate of the cyclooxygenase enzyme inhibitor seems to be dependant on the manufacturing process employed. Especially, it was judged necessary to control critical parameters like compression force, etc. to produce the dosage form compositions.

### In-Vivo Study Method

A comparative bio-availability (in vivo) study of nimesulide formulations of the present invention was carried out against Aulin® tablets (CSC PHARMACEUTICALS HANDELS GmbH) in a group of healthy humans. The aim of the study was to conduct comparative pharmacokinetic evaluation of modified release formulations containing 200 mg (referred to as ‘T-1’) of Nimesulide. The Nimesulide modified release tablets (Test composition i.e. ‘T-1’ as in example-1) was evaluated against Nimesulide conventional release tablet (Aulin® 2×100 mg immediate release tablets referred to as ‘REFERENCE’ i.e. R-1 taken at 0 hours) in healthy human volunteers, before and after food, using a, randomized, open-label, balanced, two-treatment, two period, two-sequence, multiple-dose, cross over design and relative bioavailability study. The study design involved twelve healthy human volunteers aged between 22-31 years, weighing 70.1±8 kgs with a mean BMI (Body Mass Index) of 16.9±1.9. Two studies namely fed & fasted studies were conducted by giving the formulations after heavy breakfast and fasting conditions respectively. Volunteers were abstained from caffeine intake for 24 hours before the study and during the period of study. Two study periods were used for the experiment. The dosing was conducted for 7 days in each period. One Tablet of Test product at “0” hour on each day or two tablets of Reference product at “0” hour on each day was orally administered with 240-ml of water after consuming the whole standard high fat non-vegetarian breakfast within 30 minutes. Drug analysis was done by collecting blood samples in vials through indwelling cannula/clean vein puncture throughout the study at predose with a blood sample (1×5 ml) within 1 hour prior to dosing on each day up to 7 days. The post dose samples (1×5 ml) were collected at 0.5, 1.0, 1.5, 2.0, 2.5, 3.0, 4.0, 5.0, 6.0, 9.0, 12.0, 12.5, 13.0, 13.5, 14.0, 14.5, 15.0, 16.0, 18.0, 20.0 and 24.0 hours after the administration of nimesulide tablet. The blood samples were collected in sample collection tubes containing Sodium EDTA as the anticoagulant. The plasma obtained was separated from blood by centrifugation and the samples were analyzed using HPLC for the concentration of Nimesulide. The various pharmacokinetic parameters (pK) were evaluated namely Cₘₐₓ (peak plasma concentration of the drug), Tₘₐₓ (time to reach peak plasma concentration), AUC₀₋ₜ (area under the ‘plasma concentration versus time’ curve from time=0 to time=t where ‘t’ denotes the time of last measurable concentration), AUC_{0-∞} (area under the ‘plasma concentration versus time’ curve from t=0 to time=∞, where ‘∞’ denotes infinity) and t_{1/2} (plasma elimination half life). The statistical and pharmacokinetic analyses were generated using WinNonlin® software (version 5.0). The statistical and pharmacokinetic parameters are presented in Table-1 (for fed study) and Table-2 (for fasted study) below.

TABLE 1
Comparative pharmacokinetic parameters of ‘REFERENCE’ (R-1)
and TEST composition (T-1) in the fed state:

| | |
|---|---|
| | Nimesulide |
| pK parameters | composition |

| (WinNolin Version 5.0) | R-1 | T-1 |
|---|---|---|
| Tₘₐₓ (hrs) | 2.7692 | 5.7308 |
| Cₘₐₓ (μg/ml) | 11.5392 | 7.4854 |
| AUC_{Last0-24} (μg/ml/hr) | 88.0119 | 71.9113 |
| AUC_{0-∞} (μg/ml/hr) | 89.0707 | 72.9913 |

TABLE 2
Comparative pharmacokinetic parameters of ‘REFERENCE’ (R-1)
and TEST composition (T-1) in the fasted state:

| | |
|---|---|
| | Nimesulide |
| pK parameters | composition |

| (WinNolin Version 5.0) | R-1 | T-1 |
|---|---|---|
| Tₘₐₓ (hrs) | 2.817 | 3.633 |
| Cₘₐₓ (μg/ml) | 8.634 | 3.650 |
| AUC₀₋₂₄ (μg/ml/hr) | 65.160 | 31.243 |
| AUC_{0-∞} (μg/ml/hr) | 66.570 | 32.220 |

The study showed that the TEST product (T-1) i.e. the Nimesulide Modified release composition of the present invention achieved a delayed Tₘₐₓ compared to the REFERENCE product which is a ‘immediate release’ composition in both the fasted and fed state studies. However, the Cₘₐₓ and AUC values obtained for the TEST product in both the studies indicated that the composition of the present invention produced desired plasma concentration of nimesulide for extended period of time. All the pharmacokinetic parameters evaluated in the study showed an increase in the fed state study in comparison to the fasted state study for the TEST as well as REFERENCE product indicating that presence of food in stomach might increase the plasma concentration of nimesulide in both the Modified release (MR) and Immediate release (IR) formulations.

The examples of pharmaceutical compositions given below serve to illustrate embodiments of the present invention. However, they do not intend to limit the scope of present invention.

### EXAMPLES

### Example-1

### Nimesulide Modified Release Tablet

### A) Immediate Release Layer

| S. No. | Name of Ingredient | Qty./tablet (mg) |
|---|---|---|
| 1. | Nimesulide micronized | 50.00 |
| 2. | Lactose | 87.03 |
| 3. | Croscarmellose sodium | 3.75 |
| 4. | Colloidal silicon dioxide | 3.00 |
| 5. | Maize starch | 19.55 |
| 6. | Povidone (K-30) | 3.00 |
| 7. | Docusate sodium | 3.40 |
| 8. | Ferric oxide (red) | 0.47 |
| 9. | Purified water | q.s. |
| 10. | Magnesium stearate | 0.80 |
| 11. | Croscarmellose sodium | 7.25 |
| 12. | Colloidal silicon dioxide | 2.50 |
| 13. | Povidone (K-30) | 1.25 |

### Procedure

i) Ingredients 1 to 5 were mixed together and sifted through mesh #30 sieve. Ingredient 8 was dissolved in the abovesaid mixture.
ii) Ingredients 6 and 7 were dissolved in ingredient 9 to obtain a homogeneous solution.
iii) The material of step (i) was granulated with the material of step (ii) followed by drying and sifting through mesh #16 sieve.
iv) Ingredients 10, 11, 12 and 13 were mixed together.
v) The granules of step (iii) were lubricated with the material of step (iv).

### B) Extended Release Layer

| S. No. | Name of Ingredient | Qty./tablet (mg) |
|---|---|---|
| 1. | Nimesulide micronized | 150.00 |
| 2. | Lactose | 69.75 |
| 3. | Hydroxypropyl methylcellulose (K4 MCR) | 52.50 |
| 4. | Docusate sodium | 3.00 |
| 5. | Povidone (K-30) | 3.00 |
| 6. | Colloidal silicon dioxide | 1.50 |
| 7. | Magnesium stearate | 1.50 |
| 8. | Colloidal silicon dioxide | 1.50 |
| 9. | Magnesium stearate | 1.50 |
| 10. | Povidone (K-30) | 3.00 |
| 11. | Purified water | q.s. |

### Procedure:

i) Ingredients 1 and 2 were mixed together and sifted through mesh #30 sieve.
ii) Ingredient 3 was dissolved in material of step (i)
iii) Ingredient 4 and ingredient 5 were dissolved in ingredient 11 to obtain a homogeneous solution.
iv) The material of step (ii) was granulated with the material of step (iii) followed by drying of the granules
v) Ingredients 6 and 7 are sifted together and mixed with the dried granules of step (iv).
vi) Ingredients 8, 9 and 10 were blended together.
vii) The material of step (v) was lubricated with the material obtained from step (vi).

The material obtained in step (v) of (A) and the material obtained in step (vii) of (B) were mixed together and compressed into tablet.

### Example-2

### Nimesulide Modified Release Capsule

### A) Immediate Release Fraction

| S. No. | Name of Ingredient | Qty./tablet (mg) |
|---|---|---|
| 1. | Nimesulide | 50.0 |
| 2. | Mannitol | 80.0 |
| 3. | Sodium starch glycollate | 5.0 |
| 4. | Colloidal silicon dioxide | 3.0 |
| 5. | Corn starch | 10.0 |
| 6. | Povidone (K-30) | 3.0 |
| 7. | Polysorbate 80 | 1.0 |
| 8. | Purified water | Lost in processing |
| 9. | Magnesium stearate | 1.0 |
| 10. | Croscarmellose sodium | 8.0 |

### Procedure

i) Ingredients 1 to 5 were mixed together and sifted through mesh #30 sieve.
ii) Ingredients 6 & 7 were dissolved in ingredient 8 to obtain a homogeneous solution.
iii) The material of step (i) was granulated with the material of step (ii) followed by drying and sifting through mesh #16 sieve.
iv) Ingredients 9 & 10 were sifted through mesh #40 sieve.
v) The material of step (iv) was mixed with the material of step (iii).

### B) Sustained Release Fraction

| S. No. | Name of Ingredient | Qty./tablet (mg) |
|---|---|---|
| 1. | Nimesulide | 150.0 |
| 2. | Lactose monohydrate | 40.0 |
| 3. | Methacrylate polymer | 60.0 |
| 4. | Docusate sodium | 3.0 |
| 5. | Hydroxypropyl methylcellulose | 2.5 |
| 6. | Purified water | Lost in processing |
| 7. | Colloidal silicon dioxide | 3.5 |
| 8. | Magnesium stearate | 2.0 |

### Procedure

i) Ingredients 1 to 3 were mixed together and sifted through mesh #30 sieve.
ii) Ingredients 4 & 5 were dissolved in ingredient 6 to obtain a homogeneous dispersion.
iii) The material of step (i) was granulated with the material of step (ii) followed by drying and sifting through mesh #24 sieve.
iv) Ingredients 7 & 8 were sifted through mesh #40 sieve.
v) The material of step (iv) was mixed with the material of step (iii).

The material obtained in step (v) of (A) and the material obtained in step (v) of (B) were mixed together and filled into hard gelatin capsule.

### Example-3

### Nimesulide Modified Release Minitablets Filled in Capsule

### A) Immediate Release Fraction

| S. No. | Name of Ingredient | Qty./tablet (mg) |
|---|---|---|
| 1. | Nimesulide | 50.0 |
| 2. | Mannitol | 6.5 |
| 3. | Sodium starch glycollate | 6.0 |
| 4. | Corn starch | 5.0 |
| 5. | Polysorbate 80 | 1.0 |
| 6. | Magnesium stearate | 1.5 |

### Procedure

i) Ingredients 1 to 5 were mixed together and sifted through mesh #30 sieve.
ii) Ingredient 6 was sifted through mesh #40 sieve.
iii) The material of step (i) was mixed with the material of step (ii) and compressed into minitablet.

### B) Delayed Release Fraction

| S. No. | Name of Ingredient | Qty./tablet (mg) |
|---|---|---|
| 1. | Nimesulide | 50.0 |
| 2. | Lactose monohydrate | 6.5 |
| 3. | Docusate sodium | 2.0 |
| 4. | Povidone (K-30) | 3.0 |
| 5. | Colloidal silicon dioxide | 3.0 |
| 6. | Magnesium stearate | 3.0 |
| 7. | Methacrylate polymer | 5.5 |
| 8. | Triethyl citrate | 1.5 |
| 9. | Isopropyl alcohol | Lost in processing |
| 10. | Methylene chloride | Lost in processing |

### Procedure

i) Ingredients 1 to 5 were mixed together and sifted through mesh #30 sieve.
ii) Ingredient 6 was sifted through mesh #40 sieve.
iii) The material of step (i) was mixed with the material of step (ii) and compressed into minitablet.
iv) Ingredient 7 & 8 were dispersed in a mixture of 9 & 10 and mixed.
v) The minitablets of step (iii) were coated with the material of step (iv).

### C) Sustained Release Fraction

| S. No. | Name of Ingredient | Qty./tablet (mg) |
|---|---|---|
| 1. | Nimesulide | 100.00 |
| 2. | Lactose Monohydrate | 10.0 |
| 3. | Sodium carboxymethylcellulose | 7.5 |
| 4. | Docusate Sodium | 3.00 |
| 5. | Povidone (K-30) | 3.00 |
| 6. | Purified Water | Lost in processing |
| 7. | Colloidal Silicon Dioxide | 3.00 |
| 8. | Magnesium Stearate | 3.00 |

### Procedure

i) Ingredients 1 to 3 were mixed together and sifted through mesh #30 sieve.
ii) Ingredients 4 & 5 were dissolved in ingredient 6 to obtain a homogeneous dispersion.
iii) The material of step (i) was granulated with the material of step (ii) followed by drying and sifting through mesh #18 sieve.
iv) Ingredients 7 & 8 were sifted through mesh #40 sieve.
v) The material of step (iv) was mixed with the material of step (iii) and compressed into minitablets.

The minitablets obtained in step (iii) of (A), step (v) of (B) & (C) were filled into hard gelatin capsule.

### Example-4

### Naproxen Modified Release Tablet

| S. No. | Name of Ingredient | Qty./tablet (mg) |
|---|---|---|
| 1. | Naproxen | 500.0 |
| 2. | Lactose Monohydrate | 100.0 |
| 3. | Croscarmellose Sodium | 4.0 |
| 4. | Corn Starch | 20.0 |
| 5. | Povidone (K-30) | 3.0 |
| 6. | Hydroxypropyl cellulose | 3.5 |
| 7. | Sodium lauryl sulphate | 4.5 |
| 8. | Purified Water | Lost in processing |
| 9. | Magnesium Stearate | 1.5 |

### Procedure

i) Ingredients 1 to 4 were mixed together and sifted through mesh #30 sieve.
ii) Ingredients 5, 6 & 7 were dissolved in ingredient 8 to obtain a homogeneous solution.
iii) The material of step (i) was granulated with the material of step (ii) followed by drying and sifting through mesh #24 sieve.
iv) Ingredient 9 was sifted through mesh #40 sieve.
v) The material of step (iv) was mixed with the material of step (iii) and compressed into tablet.

### Example-5

### Celecoxib Modified Release Tablet

| S. No. | Name of Ingredient | Qty./tablet (mg) |
|---|---|---|
| 1. | Celecoxib | 100.0 |
| 2. | Microcrystalline cellulose | 58.5 |
| 3. | Sodium starch glycollate | 3.0 |
| 4. | Hydroxypropyl methyl cellulose | 52.5 |
| 5. | Isopropyl alcohol | Lost in processing |
| 6. | Croscarmellose sodium | 3.0 |
| 7. | Colloidal silicon dioxide | 3.0 |
| 8. | Magnesium stearate | 3.0 |

### Procedure

i) Ingredients 1 to 3 were mixed together and sifted through mesh #30 sieve.
ii) Ingredient 4 was dissolved in ingredient 5 to obtain a homogeneous dispersion.
iii) The material of step (i) was granulated with the material of step (ii) followed by drying and sifting through mesh #24 sieve.
iv) Ingredients 6, 7 & 8 were sifted through mesh #40 sieve and mixed.
v) The material of step (iv) was mixed with the material of step (iii) and compressed into tablets.

### Example-6

### Nimesulide Sustained Release Tablet

| S. No. | Name of Ingredient | Qty./tablet (mg) |
|---|---|---|
| 1. | Nimesulide micronized | 200.0 |
| 2. | Lactose | 120.0 |
| 3. | Hydroxypropyl methylcellulose KGMCR | 50.0 |
| 4. | Carboxymethylcellulose sodium | 52.5 |
| 5. | Cremophor RH40 | 4.0 |
| 6. | Polyvinyl pyrrolidone | 8.0 |
| 7. | Magnesium stearate | 3.0 |
| 8. | Colloidal silicon dioxide | 4.0 |
| 9. | Isopropyl alcohol | Lost in processing |

### Procedure

i) Ingredients 1 to 4 were mixed together and sifted through mesh #40 sieve.
ii) Ingredient 6 was dissolved in 9 and the mixture was dissolved with 5.
iii) The material of step (i) was granulated with the material of step (ii). The granules were passed through #16 sieve followed by drying and again sifting through mesh #22 sieve.
iv) The granules of step (iii) were lubricated with 7 and 8.
v) The material of step (iv) was compressed into tablets.

1-30. (canceled)

31. A modified release pharmaceutical dosage form which comprises nimesulide or its pharmaceutically acceptable salts, esters, prodrugs, solvates, hydrates, or derivatives thereof as active agent, said nimesulide being treated with at least one release controlling polymer, wherein the dosage form provides a release of not more than about 60% of nimesulide in 1 hour and not less than about 75% of nimesulide after 12 hours when tested in accordance with the dissolution method (I) described herein employing distilled water with 2.0% sodium lauryl sulphate as the dissolution medium or in accordance with the dissolution method (II) described herein employing pH 7.0 phosphate buffer with 2.0% sodium lauryl sulphate as the dissolution medium or in accordance with the dissolution method (III) described herein employing 0.001 N hydrochloric acid with 1.0% sodium lauryl sulphate as dissolution medium.

32. A modified release pharmaceutical dosage form according to claim 31, which comprises nimesulide or its pharmaceutically acceptable salts, esters, prodrugs, solvates, hydrates, or derivatives thereof as active agent, said nimesulide being treated with at least one release controlling polymer, which when tested in a group of healthy humans achieves a mean peak plasma concentration (Cₘₐₓ) after at least about 1 hour of administration of the dosage form.

33. A modified release pharmaceutical dosage form according to claim 32, wherein the mean peak plasma concentration (Cₘₐₓ) is achieved within about 2-13 hours of administration of the dosage form.

34. A modified release pharmaceutical dosage form according to claim 31, wherein the composition when tested in vivo exhibits a mean Cₘₐₓ (peak plasma concentration) of about 0.5-30 μg/ml and/or a mean Tₘₐₓ (time to reach peak plasma concentration) of about 1-12 hours.

35. A modified release pharmaceutical dosage form according to claim 31, wherein the dosage form comprises about 5 to about 400 mg of nimesulide and at least one release controlling polymer; and wherein the said dosage form provides a mean Cₘₐₓ in the range of about 3-24 μg/ml achieved in a mean time (Tₘₐₓ) in the range of about 2-8 hours; and wherein the said dosage form provides a therapeutic effect for at least about 8 to about 24 hours after oral administration.

36. A modified release pharmaceutical dosage form comprising nimesulide as the active agent according to claim 31, wherein the said dosage form provides an in-vitro dissolution of from about 5% to about 50% of nimesulide released after 1 hour; from about 40% to about 85% of nimesulide released after 6 hours; and not less than about 70% of nimesulide released after 12 hours when tested by the USP Apparatus Type II (Paddles) at 100 rpm using 1000 ml of Distilled water with 2.0% Sodium lauryl sulphate as dissolution medium maintained at about 37±0.5° C. temperature.

37. A release pharmaceutical dosage form according to claim 31, which comprises nimesulide or its pharmaceutically acceptable salts, esters, prodrugs, solvates, hydrates, or derivatives thereof as active agent, said nimesulide being treated with at least one release controlling polymer wherein the dosage form provides a release of not more than about 60% of the cyclooxygenase enzyme inhibitor in about 1 hour when tested by USP Apparatus Type II (Paddles) at 100 rpm, using 1000 ml of dissolution medium maintained at about 37±0.5 C, wherein the dissolution medium is any one selected from pH 7.4 phosphate buffer USP or USP Simulated Intestinal Fluid or USP Simulated Gastric fluid or pH 4.5 Acetate buffer USP.

38. A modified release pharmaceutical dosage form according to claim 31, wherein the dosage form intended for once-a-day, twice-a-day or thrice-a-day administration, releases nimesulide in a desired manner so as to maintain prophylactic and/or therapeutic levels of nimesulide in the plasma for extended period of time devoid of any substantial drug related toxicity.

39. A modified release pharmaceutical dosage form according to claim 38, wherein the dosage form is intended for once-a-day administration.

40. The dosage form according to claim 39, wherein the modified release dosage form is in the extended release form, sustained release form, timed release form, pulsatile release form, prolonged release form or delayed release form.

41. The dosage form according to claim 40, wherein the modified release form is in the form of a combination of immediate release form and extended release form.

42. The dosage form according to claim 31, wherein the active agent nimesulide is in the micronized form.

43. The dosage form according to claim 31, which comprises one or more pharmaceutically acceptable carrier(s).

44. The dosage form according to claim 33, wherein the pharmaceutically acceptable carrier comprises a polymeric material selected from the group comprising pH dependent polymers; pH independent polymers; swellable polymers; hydrophilic polymers; hydrophobic polymers and/or one or more other hydrophobic materials; ionic polymers; non-ionic polymers; synthetic or natural polysaccharides and mixtures thereof.

45. The dosage form according to claim 31, which additionally comprises one or more of a gum, at least one surfactant, at least one complexing agent, antimicrobial preservative and/or antioxidant.

46. A modified release pharmaceutical dosage form according to claim 31, which is formulated into a dosage form selected from the group comprising of oral solid dosage forms, liquid dispersions, oral suspensions, gels, aerosols, ointments, creams, fast melt formulations, rapidly disintegrating formulations, mucoadhesive formulations, gastroretentive formulations, and lyophilized formulations.

47. The dosage form according to claim 46, which is in the form of a tablet or capsule.

48. A method of using the dosage form according to claim 31, for the treatment of cyclooxygenase enzyme mediated disorders and/or cyclooxygenase inhibitor indicated disorders, which comprises administrating to a subject in need thereof a pharmaceutically effective amount of nimesulide or its pharmaceutically acceptable salts, esters, prodrugs, solvates, hydrates, or derivatives thereof.

49. A method according to claim 48, for the treatment of acute painful conditions selected from post-operative trauma, pain associated with cancer, sports injuries, migraine headache, neurological pain and pain associated with sciatica and spondylitis or arthritis.

50. A modified release pharmaceutical dosage form according to claim 31, wherein the mean peak plasma concentration (Cₘₐₓ) is achieved within about 2-13 hours of administration of the dosage form.

51. A modified release pharmaceutical dosage form according to claim 32, wherein the composition when tested in vivo exhibits a mean Cₘₐₓ (peak plasma concentration) of about 0.5-30 μg/ml and/or a mean Tₘₐₓ (time to reach peak plasma concentration) of about 1-12 hours.
